(19) United States
(12) Patent Application Publication
(10) Pub. No.: US 2017/0296391 A1
(43) Pub. Date: Oct. 19, 2017

(54) Adhesive Bandage with Raised portion, Water Temperature Indicator Strip, and non Adhesive Tab

(71) Applicant: Tianna Michelle Kendall, Pullman, WA (US)

(72) Inventor: Tianna Michelle Kendall, Pullman, WA (US)

(21) Appl. No.: 15/098,667

(22) Filed: Apr. 14, 2016

(51) Int. Cl. A61F 13/00; A61F 13/02

(52) U.S. Cl. CPC A61F 13/00059; A61F 13/0233; A61F 13/025; A61F 13/00085; A61F 2013/00182; A61F 2013/00217; A61F 2013/00165

(57) ABSTRACT

Adhesive bandages/shield/cover with at least one raised non-adhesive portion which is elevated above the at least one adhesive layer with attached water temperature gauge indicator. The raised non-adhesive portion is peripherally surrounded by an adhesive layer with attached water temperature gauge indicator and is designed to protect surgical sites and cutaneous wounds as well as any other skin malady. Neither the raised non-adhesive portions of the bandage/shield/cover nor the adhesive layer with attached water temperature gauge indicator are in contact with the cutaneous layer which is the subject of the sought protection, with the adhesive layer with attached water gauge temperature indicator coming into contact only with the area surrounding the affected site which can result in the reduction or elimination of exposure to moisture and/or contaminates, and the correct water temperature can be indicated to eliminate any scalding of the skin. In addition, the surgical site or cutaneous wound is protected from force or impact. The raised non-adhesive portion may also be transparent to provide for monitoring of the surgical site or cutaneous wound while the surgical site or cutaneous wound remains protected.

### BACKGROUND

Throughout the lengthy history of medicine, a common and serious problem has been discovering new and improved ways of keeping wounds, cutaneous surgical sites, infant umbilical cords and other cutaneous maladies clean, dry and safe from water scalding which could cause serious problems in facilitating rapid healing. This is especially difficult when some aspect of the wound is raised in such a way that traditional bandages and shields cannot adequately cover it in any type of sealed way, and/or the elevated plane and differing surfaces of some wounds raise the bandage unevenly which can allow air and water to potentially make contact with the wound which is already very sensitive and delicate. In addition, the strip shape of traditional bandages and shields does not offer any sort of seal, a water temperature indicator or added protection along all the edges, particularly, as is the case in some traditional bandages and shields which have gauze in the central portion of the inner face of the bandage.

This difficulty arises, for example, in the case of newborn babies, where the remaining umbilical stump resulting from the severing of the umbilical cord is extremely delicate and prone to infection. Medical professionals advise to keep this umbilical remnant clean and dry to facilitate its eventual healing and subsequent dropping off. Keeping a newborn clean and dry is already challenging, especially with regard to the stump which is in close proximity to body parts which need to be washed regularly and at the correct water temperature to avoid other localized infection. In this way, there is a need for a solution which can keep the umbilical stump protected, dry, and safe from any skin burns during such activities such as bathing, which at the same time does not come into contact with the stump which can result in discomfort, skin scarring and possible irritation. Traditional bandages and shields are simply not designed for such tasks.

An additional cutaneous issue is trying to keep peripherally inserted central catheter lines (hereinafter, “PICC lines”) and other medical apparatuses such as accessed ports dry and safe from unhealthy elevated water temperature while at the same time allowing the patient to live a normal life in which bathing and occasional exposure to moisture is an unavoidable necessity. Traditional adhesive bandages and shields also fail in this regard as the adhesive is often not water resistant, and doesn't have a water temperature indicator. In addition, the issue of a raised surface, previously discussed, makes it difficult to have any full coverage over an uneven area without the possibility of resulting air pockets gapping and subsequent potential introduction of moisture, bacteria and/or other foreign matter. This is crucial as the skin is very fragile and sensitive. Any exposure to elevated water temperature could cause severe long term problems and scarring which could cause physical, emotional, and mental issues, particularly when there is no protective adhesive layer surrounding every edge of the bandage. In addition, traditional bandages and shields have continuous contact with the skin which can further irritate the area of contact with the bandage and prolong healing time, not to mention causing pain to the area with the potential of constant rubbing and/or contact. Along the same lines, removal of traditional bandages from cutaneous wounds or surgical sites can be extremely painful as there is a potential that the adhesive portion will have adhered to at least part of the cutaneous wound or surgical site.

The same challenge of keeping an area clean and dry is also inherent in cases where there are sutures or stitches. One is often advised that such areas be kept dry which is challenging in one's day to day life. Again, traditional bandages and shields which often have an adhesive layer which is located close to the wound site can stick to the wound resulting in discomfort and in other cases the possibility of accidental pulling and/or removal of the stitches or sutures.

Traditional bandages and shields also do not allow for monitoring of the wound site. Once a traditional bandage is applied, the cutaneous wound or surgical site beneath it is not visible as it is covered by the bandage. The unknown state of the cutaneous wound or surgical site can cause a great deal of stress and anxiety, especially in cases in which it is imperative to keep such sites completely clean and dry. In the course of bathing or other exposure, one is not able to determine if the bandage is actually successful in keeping the wound clean, dry and safe from elevated water temperature. This introduces the real and dangerous possibility of wounds being subjected and/or exposed to prolonged moisture contact which is potentially extremely damaging. In the alternative, if one is able to monitor the wound site, one is able to immediately discontinue contact with moisture should there be even an infinitesimal amount of leakage, which reduces the amount of damage which may result from prolonged exposure.

A further longstanding problem with traditional bandages and shields relates to cutaneous wound and surgical site protection. Traditional bandages and shields are generally formed completely with soft and flexible material. This material is usually lightweight and is not capable of protecting the cutaneous wound or surgical site from any external force. In this way, traditional bandages and shields expose the covered area to potential harm from force which can be extremely damaging to the cutaneous wound or surgical site as well as being painful and prolonging the healing process.

### SUMMARY

In accordance with the following disclosure, the problem of cutaneous wound or surgical site exposure and irritation can be avoided by a bandage/shield/cover with a raised non-adhesive portion which is essentially peripherally surrounded by a partially adhesive layer with an attached water temperature gauge indicator. The partially adhesive layer with an attached water temperature gauge indicator can be formed into one or many tabs which can result in various different shapes of the bandage/shield/cover which enhances the ease by which the bandage is applied and removed, while also greatly reducing the possibility of skin puckering beneath the bandage. There is also a partition of the shield that has an additional tab located on the outer layer of the shield with no adhesive which allows for easy removal of the shield. The inclusion or exclusion of tabs also allows for customization of the shield and allows the shield to better form to fit different body parts, which in turn decreases the possibility of gapping and/or lifting as a result of movement. The bandage/shield/cover can be circular or ovular in shape and can also be water resistant. A temperature gauge strip is added along the rim to allow correct water temperature for newborn so their skin will not be damaged by unsafe water temperature. With regard to the appearance of the bandage/shield/cover, it can be all or partially transparent as a means by which the covered area can be continuously monitored.

### BRIEF DESCRIPTION OF THE DRAWINGS

FIG. 1 is an environmental view depicting an application of a bandage/shield/cover according to the accompanying disclosure.

FIG. 2 is a side view of a bandage/shield/cover in accordance with the current disclosure, depicted in a first exemplary application of use.

FIG. 3 is a side view of a bandage/shield/cover in accordance with the current disclosure, depicted in a second exemplary application of use.

FIG. 4 is a bandage/shield in accordance with the current disclosure.

FIG. 5 is a plan view of the bandage/shield/cover depicted in FIG. 1.

FIG. 6 is a plan view of an alternative embodiment of the bandage/shield depicted in FIG. 1.

FIG. 7 is a plan view of a further alternative embodiment of the bandage/shield/cover depicted in FIG. 1.

FIG. 8 is a side view of one embodiment of a bandage/shield/cover in accordance with the accompanying disclosure.

FIG. 9 is a side view of another embodiment of a bandage/shield/cover in accordance with the accompanying disclosure.

### DETAILED DESCRIPTION

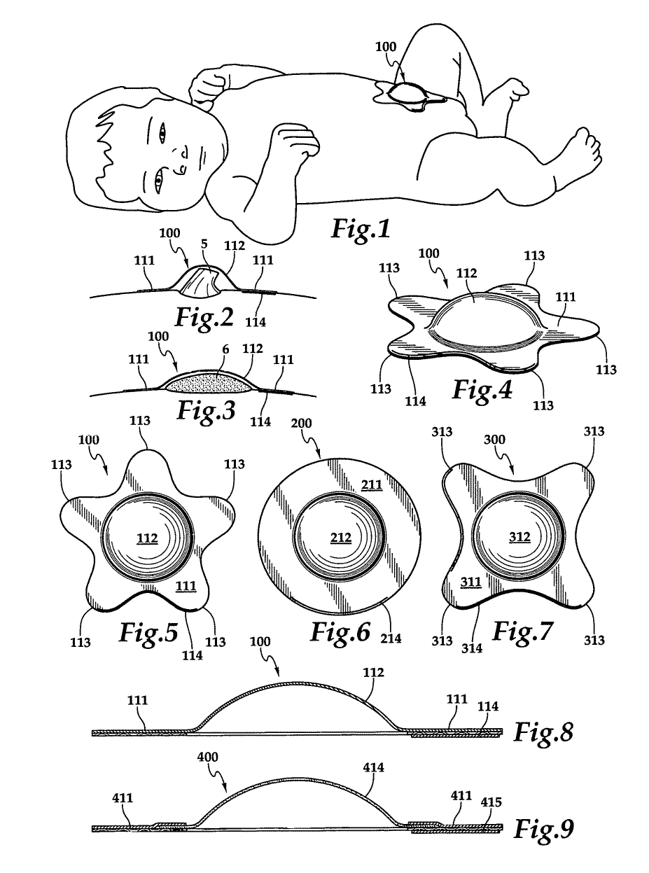

FIG. 1 depicts a preferred bandage/shield/cover 100 in accordance with the current disclosure in one possible field of use. In this particular embodiment, the shield 100 is depicted as being used as a protective layer which covers an umbilical stump 5 and the water temperature gauge indicator is flush with the cover and lays flat on the newborn's skin (114), FIG. 2. The protective layer can be water resistant and can adequately measure the temperature of the water so the newborn's skin doesn't burn (114). For example, the umbilical stump 5, FIG. 2, of a newborn baby is very delicate and must remain intact (with the infant) until the stump 5, FIG. 2, naturally drops off. This manner of use of the bandage/shield/cover 100 can be utilized as a means of protecting the stump 5, FIG. 2 during such activities which may cause potential harm to the umbilical stump 5, FIG. 2, such as bathing of the newborn.

FIG. 2 depicts a side view of a bandage/shield/cover 100 with adhesive layer 111, with the attached water temperature gage indicator (114) and raised non-adhesive portion 112. FIG. 2 depicts the bandage 100 in a field of use covering an umbilical stump 5. In this embodiment, the raised non-adhesive portion 112 is shown as transparent with the purpose of being able to view the stump 5 and monitor for leakage during activities such as bathing, and the temperature gauge strip will allow for accurate water temperature but it need not be transparent and may also be opaque. Umbilical stumps 5 are extremely sensitive and delicate and the raised non-adhesive portion 112 is not in contact with the stump as to avoid irritation and discomfort. In addition, the raised non-adhesive portion and the non adhesive tab on the outer layer of bandage/shield/cover 112 makes removal of the bandage/shield/cover 100 much easier and less dangerous as there is a greatly reduced chance that the slightly adhesive portion will come into contact with the sensitive stump which could potentially result in the bandage/shield/cover sticking to the stump 5 and pulling painfully during removal which is a common problem with prior art bandages in which the non-adhesive area lies flat against the skin. In addition, it doesn't have any water temperature gauge indicator which could cause extreme and sever skin damage, and is not elevated above the adhesive portion which surrounds it nor does it have an additional non-adhesive tab.

FIG. 3 depicts the bandage/shield/cover 100 of FIGS. 1 and 2 in another possible field of use with the adhesive layer 111 with an attached water temperature gauge indicator portion (114) and raised non-adhesive portion 112. FIG. 3 depicts the bandage/shield/cover 100 covering a raised cutaneous wound 6, although the bandage/shield/cover can be used to cover or protect any other cutaneous surface malady as well as medical apparatuses (including but not limited to PICC lines, accessed ports, sutures, stitches and other surgical sites), in the same way. In the embodiment depicted in FIG. 3, raised non-adhesive portion 112 protects the cutaneous wound 6 from exposure to moisture and other contaminates. The adhesive layer 111 and 114 can also be water resistant and temperature sensitive. The attached water temperature gauge indicator (114) allows for accurate and safe water temperature for bathing so the delicate and sensitive skin does not get damaged or scalded which can lead to further care. The raised non-adhesive portion 112 can also be transparent to allow for monitoring of the site. Again, as is depicted in FIG. 2, the raised non-adhesive portion 112 is not in contact with the wound 6 in order to avoid irritation, discomfort, and burn which is different from prior art bandages/shields/covers in which the non-adhesive gauze is not raised above the adhesive layer nor does it have an additional non adhesive tab for easy removal. In addition, because the bandage/shied/cover 100 has no contact with the cutaneous wound site 6, and the raised portion 112 is non-adhesive there is a greatly decreased risk of the raised portion of the bandage/shield/cover 100 adhering to the wound 6 during removal of the bandage. Not only does this feature decrease the chances of the pain and irritation to a patient which could result from having to remove an adhesive layer from a wound 6, but it also decreases the risk of an adhesive layer with an attached water temperature gauge indicator coming into contact with sutures or stitches and inadvertently ripping them out during the removal process (ripping out stitches or sutures during the healing process can not only be painful, but potentially dangerous as well and the raised non-adhesive portion also decreases the likelihood of this occurrence).

FIG. 4 depicts the bandage/shield/cover 100 in FIGS. 1-5 and 8 with the adhesive layer 111, an attached water temperature gauge indicator portion 114, and a raised non-adhesive portion and temperature gauge strip 112, all of which can be formed from any durable and flexible material including but not limited to silicone, rubber, or fabric all of which can be water resistant. In the preferred embodiment, the adhesive layer 111 with the attached water temperature gauge indicator 114 is completely peripherally affixed (by adhesive or other method) to the rim of the base of the raised non-adhesive portion 112 which can be dome shaped; the adhesive layer 111 with the attached water temperature gage indicator 114, extending outward from the periphery of the rim of the base of the raised non-adhesive portion 112 such that the adhesive layer 111 with the attached water temperature gauge indicator 114 is absent underneath the raised non-adhesive portion 112, such that neither the adhesive layer 111 with the attached water temperature gauge indicator gauge 114 nor the raised non-adhesive portion 112 come into contact with the cutaneous wound 6, FIG. 3, or umbilical stump, 5, FIG. 2, while the adhesive layer 111 with the attached water temperature gauge indicator 114 extends outward from the edges of the base of the raised non-adhesive portion 112 and adheres to the skin surrounding the raised non-adhesive portion 112, securing the bandage/shield/cover 100 into place.

Additionally, in the embodiment depicted in FIG. 4, several tabs 113 are shown. For example, the adhesive layer 111 with the attached water temperature gauge indicator 114 can be shaped into one or several tabs 113. These tabs 113 contribute to the ease of application and removal of the adhesive layer 111 with the attached water temperature gauge indicator 114. The tabs 113 depicted in FIG. 4 are rounded, however this need not be the case and the tabs can be presented in several different shapes. FIG. 4 shows five tabs 113, however there can be more or fewer tabs which can be either uniform in shape or variable in shape. All of the different shapes will have a temperature gage indicator and a non adhesive tab. The tabs 113 also serve another important purpose in that they allow for more ease of movement of the area, onto which the bandage/shield/cover 100 is applied. For example, it is ordinarily desirable for living beings to move and the addition of one or several tabs creates a bandage/shield/cover which has enhanced flexibility over a bandage/shield/cover of the prior art, which tends to gap, especially in the center (i.e. over a wound 6, FIG. 3, or an umbilical stump 5, FIG. 2) with different types of movement. This gapping is undesirable as it exposes the wound site to unwanted moisture and other elements including contaminates. Providing tabs 113, the number of which can be chosen to best accommodate the region of the body to which the bandage/shield/cover is applied, can greatly customize the fit of the bandage/shield/cover 100 to its particular intended application, thereby substantially reducing the risk of gapping and the resulting risk of exposure. The raised non-adhesive portion 112 is again depicted and is virtually completely peripherally bordered by the adhesive layer 111 with the attached water temperature gauge indicator 114. Depending on its fabrication, the adhesive layer 111 with the attached water temperature gauge indicator 114 may also be re-adherable such that it can be reused many times, which is especially useful in cases in which it is only adhered for a short period of time such as during bathing. The water temperature gauge indicator 114 is bonded onto the adhesive layer 111 and is re-usable many times as well.

FIG. 5 depicts the bandage/shield/cover 100 of FIGS. 1 and 4 including the adhesive layer 111, with the attached water temperature gauge 114 with the raised non-adhesive portion 112. In this embodiment, the adhesive layer, 111 with the attached water temperature gauge indicator 114 is again depicted as being shaped into several tabs 113 which aid in easing the process of removing the adhesive layer. In addition, the multiple tabs 113 allow the bandage/shield/cover 100 to be applied in such a way as to adhere more continuously with the body of a patient, so as to greatly reduce the chance of under bandage skin puckering (which can lead to discomfort and wound leakage).

The tabs 113 also provide the potential for shapes which can be appropriate and popular with children. The bandage/shield/cover 100 of FIG. 5 also depicts that the raised non-adhesive layer 112 is surrounded entirely by the adhesive layer 111 with the attached water temperature gauge indicator 114, which borders the raised portion 112 completely. For example, with respect to the bandage/shield/cover 100 of FIG. 5, there is no part of the raised non-adhesive portion 112 which creates the edge of the bandage. Encircling the elevated non-adhesive portion 112 with the adhesive layer 111 with the attached water temperature gauge indicator 114 provides heightened structural security for the bandage 100. There is essentially not one portion of the raised non-adhesive portion 112 which is not anchored by the adhesive layer 111 with the attached water temperature gauge indicator 114. The anchoring by the adhesive layer 111 with the attached water temperature gauge indicator 114 greatly reduces the chances of lifting of the bandage/shield/cover 100 or gapping of the bandage 100 which can result in the wound site 6, FIG. 3 or umbilical stump, 5 FIG. 2, being exposed to unwanted elements (such as moisture or contaminates, which are a prevalent problem with prior art strip shaped bandages/shield/cover having edges which are not anchored by any adhesive). The safety portion of the water temperature gauge indicator 114 allows the delicate and sensitive skin to stay clean and safe from any harmful, deadly, scarring situations that could occur during bathing.

FIG. 6 depicts a bandage/shield/cover 200 with an adhesive layer 211, with the attached water temperature gauge indicator 214, and raised non-adhesive layer 212. In this embodiment, the adhesive layer 211 with the attached water temperature gauge indicator 214 is rounded and has no angular edges. Oftentimes, with cutaneous wounds 6, FIG. 3 or umbilical stumps 5, FIG. 2, the surrounding area is sensitive. This embodiment is devoid of any tabs resulting in an alternate mode of removal as only one rounded edge needs to be peeled upward in order to facilitate the removal of the bandage in its entirety which reduces the potential irritation which may be caused by having to peel or lift the bandage/shield/cover at multiple sites. In this embodiment, the raised non-adhesive portion 212 can be dome shaped and peripherally attached at the rim of its base to the adhesive layer 211 with the attached water temperature gauge indicator 214 which extends radially outward from the raised non-adhesive portion 212. Neither the raised non-adhesive portion 212 which is raised above the adhesive layer 211 with the attached water temperature gauge indicator 214, nor the adhesive layer are in contact with the wound 6, FIG. 3, or umbilical stump 5, FIG. 2 which the bandage/shield/cover 200 covers. Both the raised non-adhesive portion 212 and the adhesive layer 211 with the attached water temperature gauge indicator 214 may be formed from any polymer or other material which can be flexible and water resistant. The raised non-adhesive portion 212 and the adhesive layer 211 with the attached water temperature gauge indicator 214 need not be made from the same material and the raised non-adhesive portion 212 may be reinforced in order to make it thicker than the adhesive layer 211 with the attached water temperature gauge indicator 214.

FIG. 7 depicts a bandage/shield/cover 300 with partially adhesive layer 311 and the attached water temperature gauge indicator 314 and raised non-adhesive portion 312. In this embodiment, the adhesive layer 311 with the attached water temperature gauge indicator 314 is again shaped into separate tabs 313 for easy application and removal. FIG. 7 depicts four such tabs 313 although again, there may be a greater number of tabs or a lesser number thereof. For example, as was discussed earlier, the bandage 100, 300 is highly customizable with regard to the addition of tabs 113, 313 or as in FIG. 6, the absence of any tabs. This customizable nature results in the adhesive bandage/shield/cover (100, 200, 300) being capable of securely covering various different types of wounds 6, FIG. 3, and umbilical stumps 5, FIG. 2, in addition to being able to be tailored to best fit a plethora of different body parts. For example, a bandage/shield/cover which is shaped to fit perfectly on an elbow might not be the same shape as a bandage/shield/cover which is shaped to fit perfectly on a forehead. Again, the raised non-adhesive portion 112, 212, 312 is depicted completely, peripherally surrounded by the adhesive layer with the attached water temperature gauge indicator (respectively 111, 211, 311, 114, 214, 314).

FIG. 8 depicts a side view of the bandage/shield/cover 100, FIGS. 1-5 and 8 with adhesive layer 111 with the attached water temperature gauge indicator 114 and raised non-adhesive portion 112. FIG. 8 depicts the raised non-adhesive portion 112 which allows for discontinuous contact of the bandage/shield/cover 100 with the cutaneous wound 6, FIG. 3 or umbilical stump 5, FIG. 2, which results in a decreased chance of irritation or rubbing to the cutaneous wound 6, FIG. 3 or umbilical stump 5, FIG. 2. FIG. 8 show the adhesive layer 111 with the attached water temperature gauge indicator 114 peripherally attached to the edges of the raised non-adhesive portion 112 such that the raised non-adhesive portion 112 forms a dome shape which has no contact with a wound 6, FIG. 3 or umbilical stump 5, FIG. 2 beneath it and is secured by the adhesive layer 111 with the attached water temperature gauge indicator 114 which extends outward from the raised non-adhesive portion 112.

FIG. 9 depicts another side view of a bandage/shield/cover 400, with adhesive layer 411 and the attached water temperature gauge indicator 415 with a rigid or more resistant raised portion 414. In this embodiment, the raised non-adhesive layer 414 is comprised of a rigid or non-flexible material such as plastic or other polymer which protects the wound from stronger forces or impacts as well as protecting the wound 6, FIG. 3, or umbilical stump 5, FIG. 2, from exposure to moisture or other contaminates. In this embodiment, the raised non-adhesive layer 414 is cup shaped. The adhesive layer 411 can be permanently affixed peripherally at the rim of its base by an adhesive or other means. The adhesive layer 411 with the attached water temperature gauge indicator 415 extends outward from the base of the raised non-adhesive portion 414 and the adhesive layer 411 with the attached water temperature gauge indicator 415 adheres to the skin surrounding the wound 6, FIG. 3 or umbilical stump 5, FIG. 2 site. Neither the raised non-adhesive portion 414, nor the adhesive layer 411 with the attached water temperature gauge indicator 415 are in contact with the wound 6, FIG. 3, or umbilical stump 5, FIG. 2. As in the previous embodiments, the rigid raised portion 414 is completely peripherally surrounded by the adhesive layer 411 with the attached water temperature gauge indicator 415 and can also have no tabs or several tabs. In addition, the rigid raised portion 414 can be transparent or opaque.

I claim:

1. A bandage/shield/cover comprising:
at least one raised non-adhesive portion having at least one rim; and
at least one adhesive layer; and water temperature gauge indicator; and tab with no adhesive layer
wherein said at least one adhesive layer with attached water temperature gauge indicator is peripherally affixed to said at least one rim of said at least one non-adhesive portion; and
wherein said at least one raised non - adhesive portion is raised above said at least one adhesive layer with attached water temperature gauge indicator.

2. The bandage/shield/cover of claim 1, wherein said at least one raised non-adhesive portion is dome shaped.

3. The bandage/shield/cover of claim 1, wherein said at least one adhesive layer with attached water temperature gauge indicator is shaped into at least one tab.

4. The bandage/shield/cover of claim 1, wherein the entirety of said bandage/shield is water resistant.

5. The bandage/shield/cover of claim 1, wherein said at least one raised non-adhesive portion is transparent.

6. The bandage/shield/cover of claim 1, wherein said at least one adhesive layer with attached water temperature gauge indicator is transparent.

7. The bandage/shield/cover of claim 1, wherein said at least one adhesive layer with attached water temperature gauge indicator is formed from a flexible material.

8. The bandage/shield/cover of claim 1, wherein said raised non-adhesive portion is formed from a flexible material.

9. The bandage/shield/cover of claim 1, wherein said at least one raised non-adhesive portion is formed from a rigid material.

10. The bandage/shield/cover of claim 1, wherein said at least one adhesive layer with attached water gauge temperature indicator is re - adherable.

11. The bandage/shield/cover of claim 1, wherein said at least one adhesive layer has a water temperature gauge indicator.

12. The bandage/shield/cover of claim 1, wherein said at least one non adhesive tab for easy removal.
